(19) United States
(12) Patent Application Publication
(10) Pub. No.: US 2026/0245731 A1
(43) Pub. Date: Aug. 20, 2026

(54) ATRIAL FIBRILLATION AND STROKE RISK ASSESSMENT

(71) Applicants: The Vektor Group, Inc., San Diego, CA (US); The Regents of the University of California, Oakland, CA (US)

(72) Inventors: Christopher J. T. Villongco, San Diego, CA (US); David E. Krummen, San Diego, CA (US); Christian David Marton, San Diego, CA (US)

(21) Appl. No.: 19/162,021

(22) PCT Filed: Mar. 11, 2024
(86) PCT No.: PCT/US2024/019383

§ 371 Date: Sep. 3, 2025

Related U.S. Application Data
(60) Provisional application No. 63/451,148, filed on Mar. 9, 2023.

(51) Int. Cl. G16H 50/30

(52) U.S. Cl. CPC G16H 50/30

(57) ABSTRACT

Systems and methods are provided for generating a stroke risk assessment machine learning (ML) model. A system generates an initial stroke risk ML model based on training data that includes, for each of a plurality of patients, features derived from a cardiogram of the patient and an indication of whether the patient had a stroke. The system runs simulations of electrical activity of a heart based on heart models having various morphological and physiological characteristics. The system generates simulated cardiograms based on the simulated electrical activity the simulations. The system applies the initial stroke risk ML model to features derived from a simulated cardiogram to generate a simulated stroke risk assessment. The system then generates a final stroke risk ML model based on features derived from the simulated cardiograms and the simulated stroke risk assessments.

### CROSS-REFERENCE TO RELATED APPLICATION(S)

This application claims the benefit of U.S. Pro. App. No. 63/451,148 filed on Mar. 9, 2023 and entitled “ATRIAL FIBRILLATION AND STROKE RISK ASSESSMENT,” which is hereby incorporated by reference it its entirety.

### BACKGROUND

Atrial fibrillation (AF) is the most common pathologic arrhythmia affecting millions worldwide. An ischemic stroke is a highly morbid complication of atrial fibrillation. Unfortunately, because many patients are asymptomatic during episodes of atrial fibrillation, a stroke may be the first symptom of atrial fibrillation.

Premature atrial contractions (PACs) are a common arrhythmia that may be precursor to AF and a stroke. PACs are characterized by a premature heartbeat originating in the atrium at a location other than the sinoatrial node (SA). Although PACs are often asymptomatic, a symptom may be that a person perceives a PAC as a skipped heartbeat. PACs normally do not present a significant health risk but may increase the risk for AF or a stroke. To minimize adverse effects, a PAC may be treated, for example, with beta blockers. (See, Himmelreich, Jelle C L, et al., “Frequent premature atrial contractions are associated with atrial fibrillation, brain ischemia, and mortality: a systematic review and meta-analysis,” EP Europace 21.5 (2019): 698-707, which is hereby incorporated by reference.)

Because of the potential for increased risk of an AF or stroke, it has been suggested that patients with PACs should be monitored more closely to detect symptoms of an AF or symptoms that could lead to stroke and to assess if intervention is warranted. However, intervention would be unnecessary for most patients most patients because they do not subsequently have AF and only a small percentage end up with a stroke.

### BRIEF DESCRIPTION OF THE DRAWINGS

FIG. 1 is a block diagram that illustrates components of a risk assessment system in some embodiments.

FIG. 2 is a flow diagram that illustrates the processing of the risk assessment system in some embodiments.

FIG. 3 is a flow diagram that illustrates the processing of the initial risk model generator in some embodiments.

FIG. 4 is a flow diagram that illustrates the processing of an ECG feature extractor in some embodiments.

FIG. 5 is a flow diagram of a heart model simulator in some embodiments.

FIG. 6 is a flow diagram that illustrates the processing of a final risk model generator in some embodiments.

FIG. 7 is a flow diagram of a patient risk assessor in some embodiments.

### DETAILED DESCRIPTION

An accurate assessment of an individual's increased risk of AF and/or stroke prior to or soon after the development of AF may allow for an effective intervention. The possible interventions may include:
1. Start medical and lifestyle interventions to slow atrial fibrosis to hinder the development of AF.
2. Start anticoagulation medications to reduce the risk of stroke.
3. Start antiarrhythmic drug therapy to reduce the probability of AF.
4. Perform an AF ablation to potentially suppress the AF.
5. Implant a left atrial appendage (LAA) occlusion device to prevent thrombosis from the LAA.

To assess the risk of AF and/or stroke, an AF and stroke risk assessment system provides an assessment of the risk of an AF and/or a stroke of a patient, based on a cardiogram (e.g., electrocardiogram (ECG) or vectorcardiogram (VCG)) collected from the patient. The risk assessment system includes an initial risk model generator, a heart model simulator, a final risk model generator, and a patient risk assessor. An ECG may be represented as voltage-time series, an image, or features derived from the ECG.

The initial risk model generator generates an initial AF risk machine learning (ML) model for generating an AF risk assessment given an ECG and/or an initial stroke risk ML model for generating a stroke risk assessment given an ECG. The ECG may represent a PAC episode. The heart model simulator runs simulations of electrical activity of a heart and generates a simulated ECG for each simulation. An initial risk ML model is then employed to provide an AF and/or stroke assessment for each simulated ECG. The final risk model generator generates a final AF risk ML model and/or a final stroke risk ML model using the simulated ECGs and their AF and/or stroke assessments. The patient risk assessor inputs a patient ECG and applies the final AF risk ML model and/or the final stroke risk ML model to generate a risk assessment for the patient. An initial risk ML model (e.g., for AF or stroke) and a final risk ML model (e.g., for AF or stroke) may employ different ML architectures. For example, an initial risk ML model may be based on a decision tree, and a final risk ML model may be based on neural network.

In the following, the risk assessment system is described primarily in the context of generating a stroke risk assessment. An AF risk assessment may be generated in a similar manner. Alternatively, or additionally, a combined AF/stroke risk ML model may be generated to provide multiple assessments such as based on whether a person with PACs will develop AF or have a stroke. Also, arrhythmias other than AF may lead to an increased stroke risk such as atrial flutter and other supraventricular tachycardias. The risk assessment system may generate a stroke risk ML model based on ECGs representing PACs or these arrhythmias. In addition, factors other than PACs may be precursors to AF such as heart failure with reduced ejection fraction and sick sinus syndrome. The risk assessment system may generate an AF risk ML model and a stroke risk ML model based on these factors.

The initial risk model generator performs a retrospective analysis of clinical data (e.g., based on electronic health records (EHRs)) that includes data on patients who had AF and/or a stroke and those who did not. For each patient, the initial risk model generator generates PAC features and AF features from the patients' ECGs and possibly other features derived from the patients' EHRs. The initial AF risk ML model may be trained based on the PAC features (referred to as AF risk features) along with an indication of whether the patient developed AF. The initial stroke risk ML model may be trained based on AF features and/or PAC features (referred to as stroke risk features). If trained with PAC features but without AF features, a stroke risk assessment can be provided even before AF develops. The initial risk ML model may be based on various ML techniques such as clustering or a neural network.

When the initial stroke risk ML model is based on a clustering technique (e.g., K-means clustering), the initial risk model generator generates clusters of similar stroke risk features. The initial risk model generator then assigns a stroke risk assessment to each cluster based on analysis of, for example, the percentage of the patients in the cluster who had a stroke. For example, if the percentage is 85, then the probability may be 0.85. To provide a risk assessment for a patient, the cluster to which the patient's stroke risk features are most similar is identified and outputs the risk assessment for that cluster.

When initial stroke risk ML model is based on a neural network (e.g., convolutional or recurrent), the initial stroke risk ML model is trained using training data that includes the stroke risk features labeled with whether the patient had a stroke and possibly with other data such as timing and seriousness of the stroke. To provide a risk assessment for a patient, the patient's stroke risk features (and that may include additional features of the patient) are input into the initial stroke risk ML model which outputs stroke related data such as probability of having a stroke, dying from a stroke, and/or timing of stroke.

The initial risk model generator may also generate an initial stroke risk ML model using additional features derived from additional information about each patient that may affect the risk of a stroke such as age, sex, weight, blood pressure, history of smoking, time from initial PAC to stroke, cardiac anatomy (e.g., derived from CT or x-ray images), prior ablation locations and patterns, patient physiology, and so on. The LAA morphology may be particularly relevant to a stroke risk assessment because of its hemodynamic and thrombogenic effects. (See, Masci, A., Barone, L., Dede, L., Fedele, M., Tomasi, C., Quarteroni, A. and Corsi, C., 2019. The impact of left atrium appendage morphology on stroke risk assessment in atrial fibrillation: a computational fluid dynamics study. Frontiers in physiology, 9, p. 1938, which is hereby incorporated by reference.) The LAA morphology has been classified based on their morphological complexity. For example, the classifications may be referred to as chicken wing, cauliflower, windsock, and cactus with chicken wing being the least likely to lead to an embolic event. Such classifications may be an additional feature. (See, Di Biase, L., Santangeli, P., Anselmino, M., Mohanty, P., Salvetti, I., Gili, S., Horton, R., Sanchez, J. E., Bai, R., Mohanty, S. and Pump, A., 2012. Does the left atrial appendage morphology correlate with the risk of stroke in patients with atrial fibrillation?Results from a multicenter study. Journal of the American College of Cardiology, 60(6), pp. 531-538, which is hereby incorporated by reference.) Alternatively, the risk of a thrombogenic event may be based on patient-specific modeling of hemodynamics based on a patient's LAA morphology and a stroke risk assessment made as described by Di Biase. The patient physiologies may include fibrosis, prior ablation locations and patterns, scarring, hypertrophy, dilation, inflammation, and autonomic dysregulation that relate to sustaining atrial fibrillation. The AF risk features may also include additional information derived from an ECG such as based on occurrences of multiple PACs that may include frequency of PACs, P waves per PAC, time between P waves, time between PAC and P wave, average P wave amplitude, and so on. A set of informative features may be selected using various feature selection techniques that are based on predictive power score, lasso regression, mutual information analysis, and so on.

One or more additional features may also be derived using various analysis systems. For example, an additional feature may be the source location of a current arrhythmia. A mapping system may be applied to an arrhythmia ECG to identify the source location of the arrhythmia. Such a mapping system is described in U.S. Pat. No. 10,860,754 entitled “Calibration of Simulated Cardiograms” and issued on Dec. 8, 2020, which is hereby incorporated by reference. As another example, a system may be employed to generate a metric (an additional feature) indicating AF (or AFL) burden based on analysis of ECGs collected of over a 14-day period. The ECGs may be collected using various ECG monitoring devices such as a Holter monitor, a smartwatch, a chest strap with Bluetooth connection to a smartphone, and so on. The burden may be based on the percentage of the “analyzable” time in which AF occurred. Analyzable time represents the total time in which the ECGs were collected that could be processed. For example, analyzable time would not include the time in which an electrode had an intermittent connection or when the ECG monitoring device was disconnected (e.g., while showering). As another example, a CHADS₂ score, a CHA₂DS₂-VASc score, a score based on an Atrial Fibrillation Investigators (AFI) scheme, a score based on a Stroke Prevention in Atrial Fibrillation (SPAF) Ill scheme, and/or the features used in generating the scores may be used as additional features. (See, Gage, B. F., Waterman, A. D., Shannon, W., Boechler, M., Rich, M. W. and Radford, M. J., 2001. Validation of clinical classification schemes for predicting stroke: results from the National Registry of Atrial Fibrillation. Jama, 285(22), pp. 2864-2870 and see Olesen, J. B., Torp-Pedersen, C., Hansen, M. L. and Lip, G. Y., 2012. The value of the CHA2DS2-VASc score for refining stroke risk stratification in patients with atrial fibrillation with a CHADS2 score 0-1: a nationwide cohort study. Thrombosis and haemostasis, 107(06), pp. 1172-1179, which are hereby incorporated by reference.) However, feature selection techniques can be used to assess how informative those scores would be given the clinical data to be used for training.

When the initial stroke risk ML model is based on a neural network, the initial stroke risk ML model may include a convolutional neural network that inputs an arrhythmia ECG and a neural network that inputs additional stroke risk features. The output of the convolutional neural network and neural network are input, for example, to a support vector machine which outputs a probability of stroke. The convolutional neural network, the neural network, and the support vector machine may be trained using a combined loss function.

The heart model simulator runs simulations of electrical activity of sinus rhythms PACs, and/or AFs based on different heart models. A heart model may specify various normal and abnormal cardiac morphologies including atrial characteristics (e.g., an enlarged LAA) and normal and abnormal physiologies. For each heart model, the heart simulator simulates electrical activity of the heart (e.g., 10 seconds of simulation time). For each simulation, the heart model simulator then generates an ECG based on the simulated electrical activity and identifies P waves and PAC beats within the ECG. The heart model simulator may also generate a stroke risk assessment based on the LAA morphology of the heart model used in a simulation and/or based on a computational analysis of hemodynamics in the LAA.

After the initial stroke risk ML model is generated and the simulations are complete, the final risk model generator generates a final stroke risk ML model based on the initial stroke risk ML model and the simulation mappings. For each simulation, the final risk model generator applies the initial stroke risk ML model to simulated stroke risk features derived from the simulation to generate a stroke risk assessment for the simulation. The risk assessment system effectively uses the initial stroke risk ML model to generate sufficient training data for the final stroke risk ML model. The final risk model generator may train the final stroke risk ML model using the simulated stroke risk features along with their stroke risk assessments and optionally with weights and biases initialized based on the weights and biases of the initial stroke risk ML model (e.g., transference). Alternatively, the final risk model generator may train the final stroke risk ML model using training data that includes both the simulated stroke risk features along with their assessments and the training data used to train the initial stroke risk ML model. The accuracy of the final stroke risk ML model may be evaluated by applying it to the clinical data to determine if the stroke risk assessments generated by the final stroke risk ML model are consistent with that of the clinical data.

In some embodiments, the initial stroke risk ML model may be trained only with features that are also used by the heart model simulator. If, however, the initial stroke risk ML model uses features that are not used by the heart model simulator, the risk assessment system may estimate features for each simulation. Features may be estimated using various techniques such as setting feature values based on median values, frequency of values, predicted values, and so on. The initial stroke risk ML model may also employ a KNN ML model, which does not need values for every feature.

To generate a patient risk assessment, the patient risk assessor inputs an ECG and any additional patient features and identifies stroke risk features. The final stroke risk ML model is then applied to the stroke risk features to identify the risk of a stroke. The initial stroke risk ML model may also be applied to provide a stroke risk assessment that is based solely on clinical data.

In some embodiments, the risk assessment system provides an assessment relating to the risk of developing AF or the risk of having a stroke over time such as a 1-, 5-, and 10-year risk. The training data for the initial stroke (or AF) risk ML model includes labels indicating the time between the collecting of the ECGs (and other patient characteristics) and the occurrence of a stroke or an AF diagnosis.

In some embodiments, the risk assessment system provides advice to patients or medical providers on various interventions related to development of AF and/or a stroke. The interventions include lifestyle modification (e.g., weight loss, diet, and exercise), blood pressure medication, sleep apnea therapy, anticoagulation, and/or LAA occlusion. The assessments may be provided by an intervention ML model that is trained using clinical data (e.g., collected from EHRs) of patients. The training data for the intervention ML model may include patient characteristics such as ECGs, weight, diet (e.g., vegan or vegetarian), exercise program (e.g., 150 minutes/week moderate training or 75 minutes/week high-intensity training), medical conditions (e.g., sleep apnea), medications, therapies, occurrences of PACs, AFs, or strokes, and so on, along with the timing information such as when a certain diet or medication started and ended. The risk assessment system may create a feature vector for each patient with features derived from patient characteristics. The risk assessment system also generates a label for each feature vector that indicates an outcome (e.g., occurrence or non-occurrence of a stroke). The intervention ML model may be based on various ML architectures that are described below such as a decision tree ML model. Techniques for providing assessments are described in U.S. application Ser. No. 18/587,796 entitled “Arrhythmia Assessment Machine Learning” and filed on Mar. 7, 2024, which is hereby incorporated by reference.

In some embodiments, the risk assessment system may generate a patient-specific stroke risk ML models. The initial risk model generator may identify patient training data of patients who are similar to the patient and train the initial stroke risk ML model using that training data. Because the patient training data is selected based on similarity to the patient, the initial stroke risk ML model is considered to be patient specific. In addition, the heart model simulator may run simulations based on characteristics derived from the patient that include morphological characteristics and electrophysiological characteristics, such as cardiac geometry and electrical properties. The simulations and the data derived from the simulations are also considered to be patient specific. In addition, the final stroke risk ML model is also considered to be patient specific because it is trained based on the patient-specific initial stroke risk ML model and the patient-specific simulations. The similarity between patients may be based on evaluation of certain pre-specified features. Those features may be identified manually or using a feature selection technique as described below. As another example, the patient training data may be clustered (e.g., K-means clustering) and the training data of the cluster that is most similar to the patient features may be selected for training the stroke risk ML model or AF risk ML model.

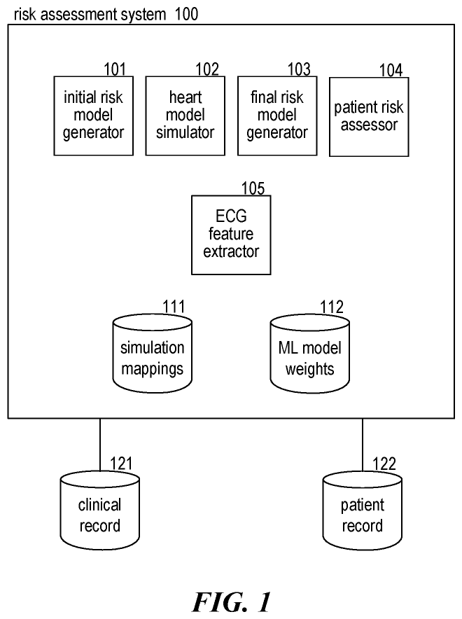

FIG. 1 is a block diagram that illustrates components of a risk assessment system in some embodiments. The risk assessment system 100 includes an initial risk model generator 101, a heart model simulator 102, a final risk model generator 103, a patient risk assessor 104, and an ECG feature extractor 105. The risk assessment system also includes a simulation mappings data store 111 and an ML model weights data store 112. The risk assessment system accesses a clinical record data store 121 and a patient record data store 122. The initial risk model generator generates an initial stroke (or AF or combined) risk ML model based on the clinical records and stores the initial model weights in the ML model weights data store. The heart model simulator runs simulations of electrical activity based on various heart models that include those that result in a PAC. The heart model simulator stores the results of the simulations in the simulation mappings data store. The final risk model generator generates a final stroke risk ML model based on the initial stroke risk ML model and the simulation mappings and stores the final model weights in the ML model weights data store. The ECG feature extractor extracts features from an ECG such as P waves.

The computing systems (e.g., network nodes or collections of network nodes) on which the risk assessment system and the other described systems may be implemented may include a central processing unit, input devices, output devices (e.g., display devices and speakers), storage devices (e.g., memory and disk drives), network interfaces, graphics processing units, communications links (e.g., Ethernet, Wi-Fi, cellular, and Bluetooth), global positioning system devices, and so on. The input devices may include keyboards, pointing devices, touch screens, gesture recognition devices (e.g., for air gestures), head and eye tracking devices, microphones for voice recognition, and so on. The computing systems may include high-performance computing systems, distributed systems, cloud-based computing systems, client computing systems that interact with cloud-based computing system, desktop computers, laptops, tablets, e-readers, personal digital assistants, smartphones, gaming devices, servers, and so on. The computing systems may access computer-readable media that include computer-readable storage mediums and data transmission mediums. The computer-readable storage mediums are tangible storage means that do not include a transitory, propagating signal. Examples of computer-readable storage mediums include memory such as primary memory, cache memory, and secondary memory (e.g., DVD), and other storage. The computer-readable storage media may have recorded on them or may be encoded with computer-executable instructions or logic that implements the risk assessment system and the other described systems. The data transmission media are used for transmitting data via transitory, propagating signals or carrier waves (e.g., electromagnetism) via a wired or wireless connection. The computing systems may include a secure crypto processor as part of a central processing unit (e.g., Intel Secure Guard Extension (SGX)) for generating and securely storing keys, for encrypting and decrypting data using the keys, and for securely executing all or some of the computer-executable instructions of the risk assessment system. Some of the data sent by and received by the risk assessment system may be encrypted, for example, to preserve patient privacy (e.g., to comply with government regulations such the European General Data Protection Regulation (GDPR) or the Health Insurance Portability and Accountability Act (HIPAA) of the United States). The risk assessment system may employ asymmetric encryption (e.g., using private and public keys of the Rivest-Shamir-Adleman (RSA) standard) or symmetric encryption (e.g., using a symmetric key of the Advanced Encryption Standard (AES)).

The one or more computing systems may include client-side computing systems and cloud-based computing systems (e.g., public or private) that each executes computer-executable instructions of the risk assessment system. A client-side computing system may send data to and receive data from one or more servers of the cloud-based computing systems of one or more cloud data centers. For example, a client-side computing system may send a request to a cloud-based computing system to perform tasks such as run a patient-specific simulation of electrical activity of a heart or train a patient-specific machine learning model. A cloud-based computing system may respond to the request by sending to the client-side computing system data derived from performing the task such as a source location of an arrhythmia. The servers may perform computationally expensive tasks in advance of processing by a client-side computing system such as training a machine learning model or in response to data received from a client-side computing system. A client-side computing system may provide a user experience (e.g., user interface) to a user of the risk assessment system. The user experience may originate from a client computing device or a server computing device. For example, a client computing device may generate a patient-specific graphic of a heart and display the graphic. Alternatively, a cloud-based computing system may generate the graphic (e.g., in a Hyper-Text Markup Language (HTML) format or an eXtensible Markup Language (XML) format) and provide it to the client-side computing system for display. A client-side computing system may also send data to and receive data from various medical devices such as an ECG monitor, an ablation therapy device, an ablation planning device, and so on. The data received from the medical devices may include an ECG, actual ablation characteristics (e.g., ablation location and ablation pattern), and so on. The data sent to a medical device may include, for example, data in a Digital Imaging and Communications in Medicine (DICOM) format. A client-side computing device may also send data to and receive data from medical computing systems of medical facilities. The data may include patient medical history data, descriptions of medical devices (e.g., type, manufacturer, and model number) that store results of procedures, and so on. The term cloud-based computing system may encompass computing systems of a public cloud data center provided by a cloud provider (e.g., Azure provided by Microsoft Corporation) or computing systems of a private server farm (e.g., operated by the provider of the risk assessment system).

The risk assessment system and the other described systems may be described in the general context of computer-executable instructions, such as program modules and components, executed by one or more computers, processors, or other devices. Generally, program modules or components include routines, programs, objects, data structures, and so on that perform tasks or implement data types of the risk assessment system and the other described systems. Typically, the functionality of the program modules may be combined or distributed as desired in various examples. Aspects of the risk assessment system and the other described systems may be implemented in hardware using, for example, an application-specific integrated circuit (ASIC) or a field programmable gate array (FPGA).

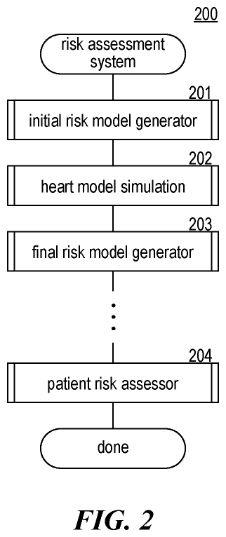

FIG. 2 is a flow diagram that illustrates the processing of the risk assessment system in some embodiments. A risk assessment system component 200 controls the overall generating of the ML models and assessment of patient risk. In block 201, the component invokes an initial risk model generator to generate the initial stroke risk ML model. In block 202, the component invokes a heart model simulator to run simulations that simulate electrical activity of the heart in which some of the simulations result in PACs. In block 203, the component invokes a final risk model generator to generate the final stroke risk ML model. In block 204, the component invokes a patient risk assessor to assess stroke risk given an ECG and features of the patient using the final stroke risk ML model.

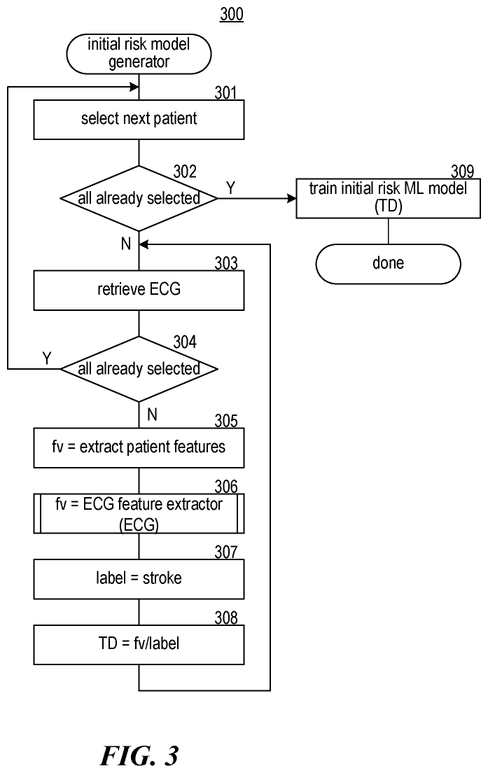

FIG. 3 is a flow diagram that illustrates the processing of the initial risk model generator in some embodiments. The initial risk model generator component 300 is invoked to generate an initial stroke risk ML model. In block 301, the component selects the next patient of the clinical data. In decision block 302, if all the patients have already been selected, then the component continues at block 309, else the component continues at block 303. In block 303, the component selects the next ECG of the selected patient. In decision block 304, if all the ECGs have already been selected, then the component loops to block 301 to select the next patient, else the component continues at block 305. In block 305, the component extracts features of the patient such as prior ablation locations and cardiac geometry. In block 306, the component invokes the ECG feature extractor passing an indication of the ECG of the patient to extract features of the ECG. The extracted features may include features for each PAC and each P wave associated with a PAC. In block 307, the component generates a label indicating whether the patient had a stroke. In block 308, the component adds the feature vector and label to the training data and loops to block 303 to select the next ECG of the selected patient. The component may add to the training data a feature vector and label for each PAC or each P wave of the ECG. The risk assessment system may assess risk given a PAC (including its P waves), an individual P wave, sequence of P waves, and so on. In block 309, the component trains the initial stroke risk ML model using the training data, and then completes.

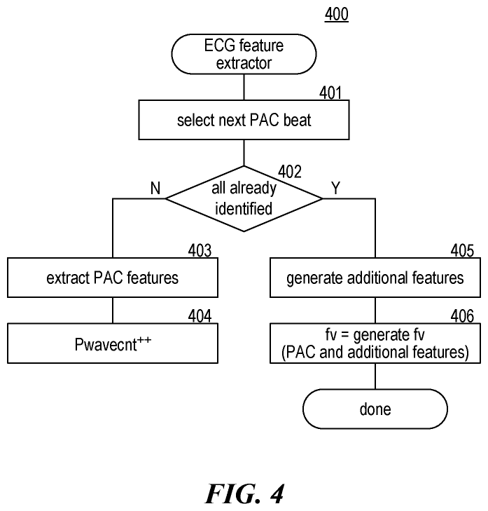

FIG. 4 is a flow diagram that illustrates the processing of an ECG feature extractor in some embodiments. The ECG feature extractor component 400 is invoked to extract features from an ECG. In block 401, the component identifies the next PAC beat within the ECG. In decision block 402, if all the PAC beats have already been identified, then the component continues at block 405, else the component continues at block 403. In block 403, the component extracts features based on the PAC beat. In block 404, the component increments various counts (e.g., PAC beat sand non-PAC beats) that may be used in generating additional features and loops to block 401 to select the next PAC beat. In block 405, the component generates additional features derived from the ECG. In block 406, the component generates a feature vector based on the PAC beat features and the additional features and then completes.

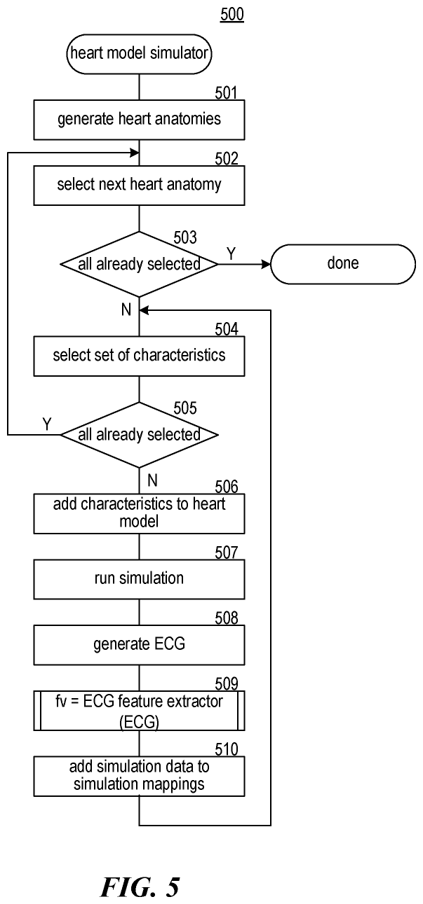

FIG. 5 is a flow diagram of a heart model simulator in some embodiments. A heart model simulator component 500 is invoked to simulate electrical activity of a heart based on various heart models. In block 501, the component generates various heart anatomies with different geometries, orientations, heart wall thickness, and so on. In block 502, the component selects the next heart anatomy. In decision block 503, if all the heart anatomies have already been selected, then the component completes, else the component continues at block 504. In block 504, the component selects the next set of characteristics such as ablation location, action potential and conduction velocity, and so on. In decision block 505, if all the sets of characteristics have already been selected, then the component loops to block 502 to select the next heart anatomy, else the component continues at block 506. In block 506, the component adds characteristics to a heart model that is based on the heart anatomy. In block 507, the component runs a simulation of electrical activity based on the heart model. In block 508, the component generates an ECG based on the simulation. In block 509, the component invokes the ECG feature extractor to extract features of the ECG. In block 510, the component adds simulation data of the simulation to the simulation mappings data store, and then loops to block 504 to select the next set of characteristics. The simulation data includes the heart anatomy, sets of characteristics, the generated ECG, the extracted ECG features, and so on.

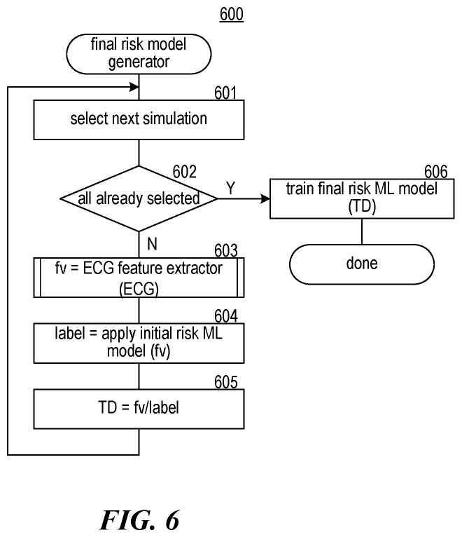

FIG. 6 is a flow diagram that illustrates the processing of a final risk model generator in some embodiments. A final risk model generator component 600 is invoked to generate a final stroke risk ML model based on the initial stroke risk ML model (or training data used to train the initial stroke risk ML model) and the simulations. In block 601, the component selects the next simulation. In decision block 602, if all the simulations have already been selected, then the component continues at block 606, else the component continues at block 603. In block 603, the component invokes the ECG feature extractor to extract stroke risk features (e.g., for each PAC or each P wave) from the ECG of the simulation or accesses features extracted by the heart model simulator. In block 604, the component generates a label by applying the initial stroke risk ML model to the stroke risk features. In block 605, the component adds the features and the label to the training data and loops to block 601 to select the next simulation. In block 606, the component trains the final stroke risk ML model using the training data. The weights of the final stroke risk ML model may be initialized to the weights of the initial risk ML model.

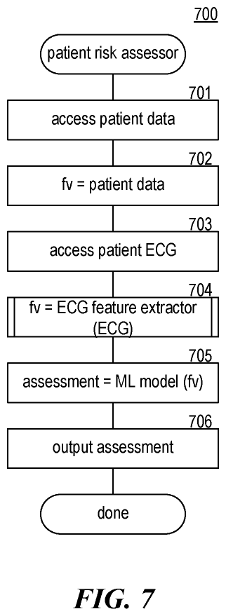

FIG. 7 is a flow diagram of a patient risk assessor in some embodiments. A patient risk assessor component 700 is invoked to assess the risk of an AF and/or stroke for a patient. In block 701, the component accesses patient data such as prior ablation locations, cardiac geometry, and so on. In block 703, the component accesses an ECG of the patient. In block 704, the component invokes an ECG feature extractor component to extract features from the ECG. In block 705, the component applies the final stroke and/or AF risk ML model to the features (PAC features and additional features) to generate an assessment of the risk of a stroke and/or AF for the patient. In block 706, the component outputs the assessment to help inform a treatment for the patient.

Although ML models and the simulations are described primarily in the context of AF and stroke risk assessment, the ML models and the simulations may be adapted to a variety of applications. The applications may relate to weather forecasting, determining properties of materials and devices, determining properties of, assessments of, and treatments of various organs (e.g., lungs, gastrointestinal tracts, and brains), and more generally analysis of physical systems. The applications may also relate to financial forecasting, employee or student assessments, message categorization, real estate pricing, and other applications in which training or using the ML models and running the simulations cannot be practically performed in the human mind. Generally, the described techniques may be employed to generate an initial ML model based on initial feature vectors with feature values of features relating to a physical process. The initial feature vectors are labeled with labeling data relating to the physical process. The techniques run simulations relating the physical process that each generates simulated feature values of features. For each simulation, the techniques generate a simulated feature vector with simulated feature values for the features. The techniques apply the initial ML model to the simulated feature vector to generate simulated labeling data for that simulated feature vector. The techniques then generate a final ML model based on the simulated feature vectors and the simulated labeling data. The final ML model is then applied to a feature vector related to the physical process to generate labeling data that is a characteristic of the physical process.

An ML model employed by the risk assessment system may be any of a variety or combination of supervised, semi-supervised, self-supervised, unsupervised, or reinforcement learning ML models including a neural network such as fully connected, convolutional, recurrent, or autoencoder neural network, a restricted Boltzmann machine, a support vector machine, a Bayesian classifier, K-means clustering, K-Nearest Neighbor (KNN), transformer, a decision tree, and so on. When the ML model is a deep neural network, the model is trained using training data that includes features derived from data and labels corresponding to the data. For example, the data may be images of ECGs with a feature being the image itself, and the labels may be a characteristic indicated by the ECGs (e.g., PAC). The training results in a set of weights for the activation functions of the layers of the deep neural network. The trained deep neural network can then be applied to new data to generate a label for that new data. When the ML model is a support vector machine, a hyper-surface is found to divide the space of possible inputs. For example, the hyper-surface attempts to split the positive examples (e.g., images PAC ECGs) from the negative examples (e.g., images of normal ECGs) by maximizing the distance between the nearest of the positive and negative examples to the hyper-surface. The trained support vector machine can then be applied to new data to generate a classification (e.g., normal sinus rhythm or PAC) for the new data. An ML model may generate values of discrete domain (e.g., classification), probabilities, and/or values of a continuous domain (e.g., regression value, classification probability).

Various techniques can be used to train a support vector machine such as adaptive boosting, which is an iterative process that runs multiple tests on a collection of training data. Adaptive boosting transforms a weak learning algorithm (an algorithm that performs at a level only slightly better than chance) into a strong learning algorithm (an algorithm that displays a low error rate). The weak learning algorithm is run on different subsets of the training data. The algorithm concentrates increasingly on those examples in which its predecessors tended to show mistakes. The algorithm corrects the errors made by earlier weak learners. The algorithm is adaptive because it adjusts to the error rates of its predecessors. Adaptive boosting combines rough and moderately inaccurate rules of thumb to create a high-performance algorithm. Adaptive boosting combines the results of each separately run test into a single, very accurate classifier. Adaptive boosting may use weak classifiers that are single-split trees with only two leaf nodes.

A neural network model has three major components: architecture, loss function, and search algorithm. The architecture defines the functional form relating the inputs to the outputs (in terms of network topology, unit connectivity, and activation functions). The search in weight space for a set of weights that minimizes the loss function is the training process. A neural network model may use a radial basis function (RBF) network and a standard or stochastic gradient descent as the search technique with backpropagation.

A convolutional neural network (CNN) has multiple layers such as a convolutional layer, a rectified linear unit (ReLU) layer, a pooling layer, a fully connected (FC) layer, and so on. Some more complex CNNs may have multiple convolutional layers, pooling layers, and FC layers. Each layer includes a neuron for each output of the layer. A neuron inputs outputs of prior layers (or original input) and applies an activation function to the inputs to generate an output.

A convolutional layer may include multiple filters (also referred to as kernels or activation functions). A filter inputs a convolutional window, for example, of an ECG image, applies weights to each pixel of the convolutional window, and outputs value for that convolutional window. For example, if the static image is 256 by 256 pixels, the convolutional window may be 8 by 8 pixels. The filter may apply a different weight to each of the 64 pixels in a convolutional window to generate the value. The convolutional layer may include, for each filter, a node (also referred to as a neuron) for each pixel of the ECG assuming a stride of one with appropriate padding. Each node outputs a feature value based on a set of weights for the filter that are learned.

An activation function has a weight for each input and generates an output by combining the inputs based on the weights. The activation function may be a rectified linear unit (ReLU) that sums the values of each input times its weight to generate a weighted value and outputs max(0,weighted value) to ensure that the output is not negative. The weights of the activation functions are learned when training a ML model. The ReLU function of max(0,weighted value) may be represented as a separate ReLU layer with a neuron for each output of the prior layer that inputs that output and applies the ReLU function to generate a corresponding “rectified output.”

A pooling layer may be used to reduce the size of the outputs of the prior layer by downsampling the outputs. For example, each neuron of a pooling layer may input 16 outputs of the prior layer and generate one output resulting in a 16-to-1 reduction in outputs.

An FC layer includes neurons that each input all the outputs of the prior layer and generate a weighted combination of those inputs. For example, if the penultimate layer generates 256 outputs and the FC layer inputs a neuron for each of classification (e.g., PAC, AF, normal sinus rhythm), each neuron inputs the 256 outputs and applies weights to generate value for its classification.

A generative adversarial network (GAN) or an attribute (attGAN) may also be used. (See, He, Z., Zuo, W., Kan, M., Shan, S. and Chen, X., 2019. Attgan: Facial attribute editing by only changing what you want. IEEE transactions on image processing, 28(11), pp. 5464-5478, which are hereby incorporated by reference.) A GAN employs a generator and discriminator and is trained using training data such as real images of objects. The generator generates generated images based on random input. The generator is trained to generate generated images that cannot be distinguished from real images. The discriminator indicates whether an input image is real or generated. The generator and discriminator are trained in parallel to learn weights. The generator is trained to generate increasingly more realistic images, and the discriminator is trained to discriminate between real images and generated images more effectively. After being trained, the generator can be used to generate ECG images that are realistic, and the discriminator can be used to discriminate between PAC and non-PAC images.

Multimodal ML combines different modalities of input data to make a prediction. The modalities may be, for example, ECG images, text (e.g., of EHRs notes), and discrete values (e.g., heart rate).

In one multimodal ML approach, referred to as “early fusion,” data of the different modalities is combined at the input stage and is then trained on the multimodal data. The training data for these modalities include a collection of sets of an image, related text, and related audio and labels. The ECG image, text, and discrete values may be used in their original form or preprocessed, for example, to reduce its dimensionality by compressing the data into byte arrays or applying a principal component analysis. Also, the resolutions of the image may be reduced. A byte array may be processed by a cross-attention mechanism to condense the bytes into a vector of a fixed size. The vectors are then used to train an ML model primarily using supervised approaches although self-supervised or unsupervised approaches may also be used.

In a second multimodal machine learning approach, data from different modalities may be kept separate at the input stage and used as inputs to different, modality-specific ML models (e.g., a CNN for image data, a transformer for text, and a recurrent neural network (RNN) for sequential data (e.g., voltage-time series)). The modality-specific ML models may be trained jointly such that information from across different modalities is combined to make predictions, and the combined (cross-modality) loss is used to adjust model weights. Alternatively, the modality-specific ML models may also be trained separately using a separate loss function for each modality. A combined ML model is then trained based on the outputs of the modality specific models. Continuing with the example, the training data for each modality-specific ML model may be based on its data along with a label. The combined ML model is then trained with the outputs of the modality-specific ML models with a final label.

Transformer machine learning was introduced as an alternative to a recurrent neural network that is both more effective and more parallelizable. (See, Vaswani, A., Shazeer, N., Parmar, N., Uszkoreit, J., Jones, L., Gomez, A. N., Kaiser, L. and Polosukhin, I., 2017. Attention is all you need. Advances in neural information processing systems, 30, which is hereby incorporated by reference.) Transformer machine learning was originally described in the context of natural language processing (NLP) but has been adapted to other applications such as image processing to augment or replace a CNN. In the following, transformer machine learning is described in the context of NLP as introduced by Vaswani.

A transformer includes an encoder whose output is input to a decoder. The encoder includes an input embedding layer followed by one or more encoder attention layers. The input embedding layer generates an embedding of the inputs. For example, if a transformer ML model is used to process a sentence as described by Vaswani, each word may be represented as a token that includes an embedding of a word and its positional information. Such an embedding is a vector representation of a word such that words with similar meanings are closer in the vector space. The positional information is based on the position of the word in the sentence.

The first encoder attention layer inputs the embeddings and the other encoder attention layers input the output from the prior encoder attention layer. An encoder attention layer includes a multi-head attention mechanism followed by a normalization sublayer whose output is input to a feedforward neural network followed by a normalization sublayer. A multi-head attention mechanism includes multiple self-attention mechanisms that each inputs the encodings of the previous layer and weighs the relevance encodings to other encodings. For example, the relevance may be determined by the following attention function:

$$Attention ( Q , K , V ) = softmax ( \frac{Q {K}^{T}}{\sqrt{{d}_{k}}} ) V$$

where Q represents a query, K represents a key, V represents a value, and d_{K} represents the dimensionality of K. This attention function is referred to as scaled dot-product attention. In Vaswani, the query, key, and value of an encoder multi-head attention mechanism is set to the input of the encoder attention layer. The multi-head attention mechanism determines the multi-head attention as represented by the following:

$$MultiHead ( Q , K , V ) = concat ( {head}_{1} , … , {head}_{8} ) {W}^{o}$$

$${head}_{i} = Attention ( {QW}_{i}^{Q} , {KW}_{i}^{K} , {VW}_{i}^{V} )$$

where W represents weights that are learned during training. The weights for the feedforward networks are also learned during training. The weights may be initialized to random values. A normalization layer normalizes its input to a vector having a dimension as expected by the next layer or sub-layer.

The decoder includes an output embedding layer, decoder attention layers, a linear layer, and a softmax layer. The output embedding layer inputs the output of the decoder shifted right. Each decoder attention layer inputs the output of the prior decoder attention layer (or the output embedding layer) and the output of the encoder. The embedding layer is input to the decoder attention layer, the output of the decoder attention layer is input the linear layer, and the output of the linear layer is input to the softmax layer which outputs probabilities. A decoder attention layer includes a decoder masked multi-head attention mechanism followed by a normalization sublayer, a decoder multi-head attention mechanism followed by a normalization sublayer, and a feedforward neural network followed by a normalization sublayer. The decoder masked multi-head attention mechanism masks the input so that predictions for a position are only based on outputs for prior positions. A decoder multi-head attention mechanism inputs the normalized output of the decoder masked multi-head attention mechanism as a query and the output of the encoder as a key and a value. The feedforward neural network inputs the normalized output of the decoder multi-head attention mechanism. The normalized output of the feedforward neural network is the output of that multi-head attention layer. The weights of the linear layer are also learned during training.

After being trained, a sentence (e.g., text of EHRs) may be input to encoder to generate an encoding of the sentence that is input to the decoder. Initially, the output of the decoder that is input to the decoder is set to null. The decoder then generates an output based on the encoding and the null input. The output of the decoder is appended to the decoder's current input, and the decoder generates a new output. This decoding process is repeated until the encoder generates a termination symbol.

Although initially developed to process sentences, transformers have been adapted for image recognition. The input a decoder of a transformer may be a representation of fixed-size patches of the image. (See, Dosovitskiy, A., Beyer, L., Kolesnikov, A., Weissenborn, D., Zhai, X., Unterthiner, T., Dehghani, M., Minderer, M., Heigold, G., Gelly, S. and Uszkoreit, J., 2020. An image is worth 16×16 words: Transformers for image recognition at scale. arXiv preprint arXiv:2010.11929, which is hereby incorporated by reference.) The representation of a patch may be, for each pixel of the patch, an encoding of its row, column, and color. The output of the encoder is fed into neural network to generate a classification of the image such as PAC for an ECG image.

The risk assessment system may also employ a state space model (SSM) to generate a latent representation of an ECG. An example of an SSM is S4 as described in Gu, A. and Dao, T., 2023. Mamba: Linear-time sequence modeling with selective state spaces. arXiv preprint arXiv:2312.00752 (Mamba), which is hereby incorporated by reference. Mamba provides a unique selection mechanism that adapts structured SSM parameters based on the input to selectively focus on relevant information within sequences, effectively filtering out less pertinent information. Mamba integrates SSM with multi-layer perceptron (MLP) blocks to support sequence modeling for sequential data such as voltage-time series of an ECG.

Fine-tuning learning is an ML technique to train an ML model by leveraging a previously trained ML model. For example, if an arrhythmia ML model has been trained to recognize whether an ECG image represents an arrhythmia, fine tuning may be used to train a PAC ML model to recognize whether an ECG image represents a PAC. The inner layers of the PAC ML model have weights that are the same as the inner layers of the arrhythmia ML model. The input layer and the output layer of the PAC ML model are trained using ECG images labeled as PAC or not. Thus, the training process need only learn the weights for the input layer and the output layer. In the PAC case, the rationale for using fine tuning is that, since PAC is one type arrhythmia, the weights of the inner layers of the arrhythmia ML model and a separately trained PAC ML model are similar. However, the input layer and the output layer of the PAC ML model need to be trained to account for the characteristics specific to a PAC. Moreover, the fine tuning may be based on a smaller set of training data than used for training the arrhythmia ML model.

An unsupervised ML technique trains an ML model using unlabeled training data. K-means clustering is an example ML technique. Given feature vectors representing the training data, K-means clustering clusters the feature vectors into cluster of similar feature vectors. With K-means clustering, the number of clusters may be predefined. For example, the risk assessment system may employ four clusters (K=3). An example training technique initially randomly places a feature vector in each cluster. The training then repeatedly calculates a mean feature vector of each cluster, selects a feature vector not in a cluster, identifies the cluster whose mean is most similar, adds the feature vector to that cluster, and moves the feature vectors already in the clusters to the cluster with the most similar mean. Similarity may be determined, for example, based on Pearson similarity, cosine similarity, and so on. The training ends when all the feature vectors have been added to a cluster. The cluster or clusters containing feature vectors for that represent PAC ECGs and non-PAC ECGs are identified, and the corresponding classification (e.g., PAC ECGs) that may be determined manually is associated with each cluster. To classify an ECG, a feature vector is generated based on the ECG, the cluster with a mean that is most similar to that feature vector is identified, and the ECG is assigned the classification of that cluster.

Self-supervised learning is an ML technique that is based on unlabeled training data. Initially, self-supervised learning augments the training data to generate additional training data to generate sets of training data that are similar. For example, if the training data is voltage-time series of ECGs, the self-supervised learning generates, for each voltage-time series, voltage-time series of with varied timings and voltages. An ML model may have an encoder layer, pretext task layer, and contrastive learning. The encoder layer may encode a voltage-time series into a latent vector. The pretext task layer includes weights for grouping voltage-time series into different clusters based on their differences using contrastive learning. Contrastive learning employs a loss function for contrasting the voltage-time series and adjusting the weights of the encoder and the pretext task layer. This approach is similar to K-means clustering but based on contrast rather than similarity. The weights of the pretext layer can be used as initial weights of a primary task such as a stroke risk ML model which can be trained using labeled training data. Self-supervised learning may be performed on multimodal data as described above.

An ML decision tree defines information (e.g., stroke risk) that is associated with patients that have certain characteristics. An ML decision may have the same form as a manually generated decision tree. However, the feature associated with each decision node of an ML decision tree may be selected automatically based on analysis of the training data. Each decision node (i.e., non-leaf node) of a decision tree corresponds to a feature, and each branch from a decision node may correspond to a value or range of values for the feature of that decision node. For example, a decision node corresponding to blood pressure may have branches for low, normal, high, and very high, and a decision node corresponding to AF may have branches for <1 year, 1-5 years, and >5 years since first detected. The leaf nodes (assessment nodes) of the decision tree may indicate the AF or stroke risk assessments. The assessment of a leaf node is intended for a patient with feature values that match the feature values of the features along the path from the root node to that leaf node. Separate decision trees may be employed to generate different assessments such as PAC risk or stroke risk. Although a single decision tree for different types of assessments could be used, the features that are important for one type of assessment may not be the same as the features that are important for another type of assessment. For example, weight may be important in a stroke risk assessment but relatively unimportant for a PAC risk assessment.

For an ML decision tree, an entropy score may be used by a ML decision tree generator to select the feature to be associated with each decision node. The entropy score for a possible feature for a decision node is based on the distribution of its values in node feature vectors for that node. A node feature vector for a decision node has the values of the branches along the path from the root node to that decision node. If a first possible feature for a decision node has an equal number of node feature vectors for each value, the entropy for the first possible feature is considered to be high. In contrast, if a second possible feature has node feature vectors for which 75% have the same value, then the entropy for the second possible feature is considered to be low. In such a case, the ML decision tree generator would select the second possible feature for that decision node. The ML decision tree generator may also analyze features with continuous values to identify cut points that tend to minimize entropy. For example, as mentioned above, heart rate may be categorized as bradycardia (<60), normal (60-100), or tachycardia (>100). However, since heart rate is a continuous value, the risk assessment system may employ techniques to identify cut points (other than 60 and 100) that would reduce the entropy. For example, a single cut point of 80 or cut points of 50 and 90 may tend to minimize entropy. Techniques for identifying cut points are described in Fayyad, U. M. and Irani, K. B., 1992. On the handling in decision tree of continuous-valued attributes generation. Machine Learning, 8, pp. 87-102, which is hereby incorporated by reference.

An ML decision tree generator may employ a depth-first, recursive algorithm to build the ML decision tree. The ML decision tree generator may employ a path termination criterion to determine when to terminate a path. The path termination criterion may be, for example, when the percentage of node feature vectors that are associated with the same assessment is above a threshold percentage. For example, if 80% of the node feature vectors have an assessment of PAC, the ML decision tree generator may add a leaf node that indicates that 80% of cohort patients has a stroke (e.g., within five years). Other termination criteria may be that the number of node feature vectors is below a threshold number, or the path has reached a maximum depth. In such cases, the ML decision tree generator may add a leaf node that indicates that an assessment cannot be provided or that the assessment has a low confidence.

To provide an assessment for a patient, a feature vector is generated for the patient based on the patient's electronic health records and/or from questions answered by the patient or medical provider. The values of the features of the patient feature vector are used to identify the path that the patient feature vector matches. The assessment is based on the leaf node of that path.

In some embodiments, a KNN model provides information relating to a patient. The training data for a KNN model may be training feature vectors (e.g., ECG images) and a label for each feature vector indicating information relating to a patient (e.g., PAC) having the values of the features of that feature vector. A KNN model may be used without a training phase that is without learning weights or other parameters to represent the training data. In such a case, the patient feature vector is compared to the training feature vectors to identify a number (e.g., represented by the “K” in KNN) of similar training feature vectors. Once the number of similar training feature vectors are identified, the labels associated with the similar training feature vectors are analyzed to provide information for the patient. The labels of the training feature vectors that are more similar to a patient feature vector may be given a higher weight than those that are less similar. For example, if k is 10 and four training feature vectors are very similar and six are less similar, similarity weights of 0.9 may be assigned to the very similar training feature vectors and 0.2 to the less similar. If three of the four and one of the six have the same information, then the information for the patient is primarily based on that information even though most of the 10 have different information. Conceptually, training feature vectors that are very similar are closer to the entity feature vector in a multi-dimensional space of features and a similarity weight is based on distance between the feature vectors. Various techniques may be employed to calculate a similarity metric indicating similarity between a patient feature vector and a training feature vector such as dot product, cosine similarity, Pearson correlation, and so on.

If the number of training feature vectors is large, various techniques may be employed to effectively “compress” the training data during a training phase. For example, a clustering technique may be employed to identify clusters of training feature vectors that are similar and have the same label. A training feature vector may be generated for each cluster (e.g., one from the cluster or one based on mean values for the features) as a cluster feature vector and assign a cluster weight to it based on number of training feature vectors in the cluster.

The ML models may input a feature vector of one or more features derived from an ECG. The features may include an imagen, a voltage-time series specifying voltages and time increments of the ECG, images and time-voltage series of portions of the ECG (e.g., T-Q interval), length in seconds of various intervals (e.g., P wave), maximum, minimum, mean, and variance of voltages of portions of the ECG, a maximal vector and angle of the vector of VCG derived from the ECG, location of a peak (Q peak) or zero crossing relative to a maximum peak (T peak) in an interval, and so on. The features used by an ML model may be manually or automatically selected. An assessment of which features may be useful in providing an accurate output for a ML model are referred to as informative feature. The assessment of which features are informative may be based on various feature selection techniques such as a predictive power score, a lasso regression, a mutual information analysis, and so on.

The features may also be latent vectors generated using an ML model such as an autoencoder. For example, an autoencoder may be trained using ECG images. In such a case, when an ECG image is input into the trained autoencoder, the latent vector that is generated is a feature vector that represents the ECG image. That feature vector can be input into another trained ML model such as a neural network or support vector machine to generate an output. When training the other ML model, for example, to classify an ECG as representing a PAC, the training ECG images are input to the autoencoder to generate training feature vectors that are labeled as being PAC or not. The other ML model is then trained using the labeled feature vectors. The autoencoder may be trained using the training ECG images or may have been previously trained using a collection of ECG images. Rather than pre-training an autoencoder, only the encoder of the autoencoder that generates the latent vector (and not the decoder) may be trained in parallel with the other ML model using a combined loss function. In such a case, no autoencoding is performed. Rather the latent vector represents features of an ECG image that are particularly relevant to generating the output of the other ML model. Such an ML architecture may be used, for example, when the other ML model (e.g., transformer) is not designed to process ECG images directly.

The following paragraphs describe various aspects of the risk assessment system. An implementation of the risk assessment system may employ any combination or sub-combination of the aspects and may employ additional aspects. The processing of the aspects may be performed by one or more computing systems with one or more processors that execute computer-executable instructions that implement the aspects and that are stored on one or more computer-readable storage mediums.

In some aspects, the techniques described herein relate to one or more computing systems for providing a stroke risk assessment for a patient, the one or more computing systems including: one or more computer-readable storage mediums that store computer-executable instructions for controlling the one or more computing systems to: generate an initial stroke risk machine learning (ML) model based on training data that includes, for each of a plurality of patients, features derived from a cardiogram of the patient and an indication of whether the patient had a stroke; for each of a plurality of simulations, run a simulation of electrical activity of a heart based on a heart model having a heart anatomy and electrical characteristics; generate a simulated cardiogram based on the simulated electrical activity; and apply the initial stroke risk ML model to features derived from the simulated cardiogram to generate a stroke risk assessment; and generate a final stroke risk ML model based on features derived from the simulated cardiograms and the generated stroke risk assessments; and one or more processors for controlling the one or more computing systems to execute one or more of the computer-executable instructions. In some aspects, the techniques described herein relate to one or more computing systems wherein the instructions further include computer-executable instructions to receive a patient cardiogram and apply the final stroke risk ML model to features derived from the patient cardiogram to generate a stroke risk assessment for the patient. In some aspects, the techniques described herein relate to one or more computing systems wherein the cardiogram is an electrocardiogram. In some aspects, the techniques described herein relate to one or more computing systems wherein a feature is derived from one or more premature atrial contraction beats of a cardiogram. In some aspects, the techniques described herein relate to one or more computing systems wherein a feature is a source location of an arrhythmia that is identified by a mapping system that inputs a cardiogram and outputs a source location. In some aspects, the techniques described herein relate to one or more computing systems wherein a feature is derived from occurrences of multiple premature atrial contraction beats. In some aspects, the techniques described herein relate to one or more computing systems wherein at least some of the patients had an arrhythmia. In some aspects, the techniques described herein relate to one or more computing systems wherein the arrhythmia is atrial fibrillation. In some aspects, the techniques described herein relate to one or more computing systems wherein the arrhythmia is an atrial flutter. In some aspects, the techniques described herein relate to one or more computing systems wherein some of the features are based on hemodynamics. In some aspects, the techniques described herein relate to one or more computing systems wherein the hemodynamics are based on morphology of the left atrial appendage. In some aspects, the techniques described herein relate to one or more computing systems wherein a stroke risk assessment related to probability of having a stroke. In some aspects, the techniques described herein relate to one or more computing systems wherein the initial stroke risk ML model and the final stroke risk ML model additionally generate an atrial fibrillation risk assessment. In some aspects, the techniques described herein relate to one or more computing systems wherein the plurality of patients are selected based on similarity to a candidate whose stroke risk assessment is to be generated by the final stroke risk ML model. In some aspects, the techniques described herein relate to one or more computing systems wherein the simulations are based on morphological characteristics and electrophysiological characteristics of a candidate whose stroke risk assessment is to be generated by the final stroke risk ML model.

In some aspects, the techniques described herein relate to a method performed by one or more computing systems for providing a risk assessment associated with an arrhythmia, the method including: accessing a risk assessment machine learning (ML) model that is generated based on training data that includes feature vectors derived from simulations and labels relating a risk assessment that are generated by an initial risk assessment ML model, wherein a simulation simulates electrical activity of a heart, wherein a feature is derived from a simulated cardiogram, and wherein the initial risk assessment ML model generates a risk assessment based on a feature vector and is generated using training data that is not derived from the simulations; and receiving a patient cardiogram of a patient; generating a patient feature vector that includes a feature value of a feature that is derived from the patient cardiogram; and applying the risk assessment ML model to the patient feature vector to generate a risk assessment for the patient. In some aspects, the techniques described herein relate to a method wherein the risk assessment relates to risk of a stroke. In some aspects, the techniques described herein relate to a method wherein a feature relates to atrial fibrillation. In some aspects, the techniques described herein relate to a method wherein a feature relates to a premature atrial contraction. In some aspects, the techniques described herein relate to a method wherein the risk assessment relates to risk of an atrial fibrillation. In some aspects, the techniques described herein relate to a method wherein a feature relates to a premature atrial contraction. In some aspects, the techniques described herein relate to a method wherein the one or more computing systems are server computing systems and the patient cardiogram is received from a client application of a patient device. In some aspects, the techniques described herein relate to a method wherein the patient device is a smartphone or a smartwatch. In some aspects, the techniques described herein relate to a method wherein the one or more computing systems are cloud-based computing systems.

In some aspects, the techniques described herein relate to a method performed by one or more computing system for generating a machine learning (ML) model, the method including: generating an initial ML model based on initial feature vectors with feature values of features, the initial feature vectors labeled with labeling data; running a plurality of simulations that each generates simulated feature values of features; for each of a plurality of simulations, generating a simulated feature vector with simulated feature values for the features; and applying the initial ML model to the simulated feature vector to generate simulated labeling data for that simulated feature vector; and generating a final ML model based on the simulated feature vectors and the simulated labeling data. In some aspects, the techniques described herein relate to a method wherein the final ML model is a decision tree. In some aspects, the techniques described herein relate to a method wherein the final ML model is a deep neural network. In some aspects, the techniques described herein relate to a method wherein the simulated labeling data relates to a stroke risk assessment and a feature is derived from a cardiogram. In some aspects, the techniques described herein relate to a method wherein the feature derived from a cardiogram relates to a premature atrial contraction. In some aspects, the techniques described herein relate to a method wherein the feature derived from a cardiogram relates to atrial fibrillation.

In some aspects, the techniques described herein relate to one or more computing systems for generating a machine learning (ML) model, the one or more computing systems including: one or more computer-readable storage mediums that store computer-executable instructions for controlling the one or more computing systems to: access a first ML model that, when applied to feature vector generates a label for that feature vector, the feature vector having features with feature values; access simulated feature vectors with features having simulated feature values; for each of a plurality of simulated feature vectors, apply the first ML model to that simulated feature vector to generate a simulated label; and generate a second ML model that, when applied to a feature vector, generates a label for that feature vector, the second ML model generated based on the simulated feature vectors and the simulated feature values; and one or more processors for controlling the one or more computing systems to execute one or more of the computer-executable instructions. In some aspects, the techniques described herein relate to one or more computing systems wherein the computer-executable instructions further control the one or more computing systems to: receive a target feature vector; apply the second ML model to the target feature vector to generate a target label; and output an indication of the target label. In some aspects, the techniques described herein relate to one or more computing systems wherein a feature relates to a premature atrial contraction and a label relates to a stroke risk assessment. In some aspects, the techniques described herein relate to one or more computing systems wherein a feature relates to an atrial fibrillation and a label relates to a stroke risk assessment. In some aspects, the techniques described herein relate to one or more computing systems wherein a feature relates to a premature atrial contraction and a label relates to an atrial fibrillation risk assessment. In some aspects, the techniques described herein relate to one or more computing systems wherein a label is a probability relating to a stroke risk assessment. In some aspects, the techniques described herein relate to one or more computing systems wherein the stroke risk assessment is based on presence of an arrhythmia. In some aspects, the techniques described herein relate to one or more computing systems wherein a feature relates to a premature atrial contraction and a label relates to a stroke risk assessment and the first ML model is trained using training data derived from electronic health records of patients.

All documents incorporated by reference are incorporated in their entirety for the full extent of their disclosures. In the event of inconsistencies between the language in this document and any incorporated-by-reference document, the language in the incorporated-by-reference document should be considered supplementary to that of this document and the language in this document controls.

Although the subject matter has been described in language specific to structural features and/or acts, it is to be understood that the subject matter defined in the appended claims is not necessarily limited to the specific features or acts described above. Rather, the specific features and acts described above are disclosed as example forms of implementing the claims.

I/We claim:

1. One or more computing systems for providing a stroke risk assessment for a patient, the one or more computing systems comprising:
one or more computer-readable storage mediums that store computer-executable instructions for controlling the one or more computing systems to:
generate an initial stroke risk machine learning (ML) model based on training data that includes, for each of a plurality of patients, features derived from a cardiogram of the patient and an indication of whether the patient had a stroke;
for each of a plurality of simulations,
run a simulation of electrical activity of a heart based on a heart model having a heart anatomy and electrical characteristics;
generate a simulated cardiogram based on the simulated electrical activity; and
apply the initial stroke risk ML model to features derived from the simulated cardiogram to generate a stroke risk assessment; and
generate a final stroke risk ML model based on features derived from the simulated cardiograms and the generated stroke risk assessments; and
one or more processors for controlling the one or more computing systems to execute one or more of the computer-executable instructions.

2. The one or more computing systems of claim 1 wherein the instructions further include computer-executable instructions to receive a patient cardiogram and apply the final stroke risk ML model to features derived from the patient cardiogram to generate a stroke risk assessment for the patient.

3. The one or more computing systems of claim 1 wherein the cardiogram is an electrocardiogram.

4. The one or more computing systems of claim 1 wherein a feature is derived from one or more premature atrial contraction beats of a cardiogram.

5. The one or more computing systems of claim 1 wherein a feature is a source location of an arrhythmia that is identified by a mapping system that inputs a cardiogram and outputs a source location.

6. The one or more computing systems of claim 5 wherein a feature is derived from occurrences of multiple premature atrial contraction beats.

7. The one or more computing systems of claim 1 wherein at least some of the patients had an arrhythmia.

8. The one or more computing systems of claim 7 wherein the arrhythmia is atrial fibrillation.

9. The one or more computing systems of claim 7 wherein the arrhythmia is an atrial flutter.

10. The one or more computing systems of claim 1 wherein some of the features are based on hemodynamics.

11. The one or more computing systems of claim 10 wherein the hemodynamics are based on morphology of the left atrial appendage.

12. The one or more computing systems of claim 1 wherein a stroke risk assessment related to probability of having a stroke.

13. The one or more computing systems of claim 1 wherein the initial stroke risk ML model and the final stroke risk ML model additionally generate an atrial fibrillation risk assessment.

14. The one or more computing systems of claim 1 wherein the plurality of patients are selected based on similarity to a candidate whose stroke risk assessment is to be generated by the final stroke risk ML model.

15. The one or more computing systems of claim 1 wherein the simulations are based on morphological characteristics and electrophysiological characteristics of a candidate whose stroke risk assessment is to be generated by the final stroke risk ML model.

16. A method performed by one or more computing systems for providing a risk assessment associated with an arrhythmia, the method comprising:
accessing a risk assessment machine learning (ML) model that is generated based on training data that includes feature vectors derived from simulations and labels relating a risk assessment that are generated by an initial risk assessment ML model, wherein a simulation simulates electrical activity of a heart, wherein a feature is derived from a simulated cardiogram, and wherein the initial risk assessment ML model generates a risk assessment based on a feature vector and is generated using training data that is not derived from the simulations; and
receiving a patient cardiogram of a patient;
generating a patient feature vector that includes a feature value of a feature that is derived from the patient cardiogram; and
applying the risk assessment ML model to the patient feature vector to generate a risk assessment for the patient.

17. The method of claim 16 wherein the risk assessment relates to risk of a stroke.

18. The method of claim 17 wherein a feature relates to atrial fibrillation.

19. The method of claim 17 wherein a feature relates to a premature atrial contraction.

20. The method of claim 16 wherein the risk assessment relates to risk of an atrial fibrillation.

21. The method of claim 20 wherein a feature relates to a premature atrial contraction.

22. The method of claim 20 wherein the one or more computing systems are server computing systems and the patient cardiogram is received from a client application of a patient device.

23. The method of claim 22 wherein the patient device is a smartphone or a smartwatch.

24. The method of claim 22 wherein the one or more computing systems are cloud-based computing systems.

25. A method performed by one or more computing system for generating a machine learning (ML) model, the method comprising:
generating an initial ML model based on initial feature vectors with feature values of features, the initial feature vectors labeled with labeling data;
running a plurality of simulations that each generates simulated feature values of features;
for each of a plurality of simulations,
generating a simulated feature vector with simulated feature values for the features; and
applying the initial ML model to the simulated feature vector to generate simulated labeling data for that simulated feature vector; and
generating a final ML model based on the simulated feature vectors and the simulated labeling data.

26. The method of claim 25 wherein the final ML model is a decision tree.

27. The method of claim 25 wherein the final ML model is a deep neural network.

28. The method of claim 25 wherein the simulated labeling data relates to a stroke risk assessment and a feature is derived from a cardiogram.

29. The method of claim 28 wherein the feature derived from a cardiogram relates to a premature atrial contraction.

30. The method of claim 28 wherein the feature derived from a cardiogram relates to atrial fibrillation.

31. One or more computing systems for generating a machine learning (ML) model, the one or more computing systems comprising:
one or more computer-readable storage mediums that store computer-executable instructions for controlling the one or more computing systems to:
access a first ML model that, when applied to feature vector generates a label for that feature vector, the feature vector having features with feature values;
access simulated feature vectors with features having simulated feature values;
for each of a plurality of simulated feature vectors, apply the first ML model to that simulated feature vector to generate a simulated label; and
generate a second ML model that, when applied to a feature vector, generates a label for that feature vector, the second ML model generated based on the simulated feature vectors and the simulated feature values; and
one or more processors for controlling the one or more computing systems to execute one or more of the computer-executable instructions.

32. The one or more computing systems of claim 31 wherein the computer-executable instructions further control the one or more computing systems to:
receive a target feature vector;
apply the second ML model to the target feature vector to generate a target label; and
output an indication of the target label.

33. The one or more computing systems of claim 31 wherein a feature relates to a premature atrial contraction and a label relates to a stroke risk assessment.

34. The one or more computing systems of claim 31 wherein a feature relates to an atrial fibrillation and a label relates to a stroke risk assessment.

35. The one or more computing systems of claim 31 wherein a feature relates to a premature atrial contraction and a label relates to an atrial fibrillation risk assessment.

36. The one or more computing systems of claim 31 wherein a label is a probability relating to a stroke risk assessment.

37. The one or more computing systems of claim 36 wherein the stroke risk assessment is based on presence of an arrhythmia.

38. The one or more computing systems of claim 31 wherein a feature relates to a premature atrial contraction and a label relates to a stroke risk assessment and the first ML model is trained using training data derived from electronic health records of patients.
